(19) United States
(12) Patent Application Publication
(10) Pub. No.: US 2011/0195413 A1
(43) Pub. Date: Aug. 11, 2011

(54) Integrated Method for Enriching and Detecting Rare Cells from Biological Body Fluid Sample

(75) Inventor: Ping Lin, Beijing (CN)

(73) Assignee: CYTTEL BIOSCIENCES CO., LTD-BEIJING, Beijing (CN)

(21) Appl. No.: 13/062,144

(22) PCT Filed: Apr. 10, 2009
(86) PCT No.: PCT/CN09/71231

§ 371 Date: Mar. 3, 2011

(30) Foreign Application Priority Data
May 20, 2008 (CN) 200810097889.4

(51) Int. Cl. C12Q 1/68; C12M 1/00; C12N 5/00

(52) U.S. Cl. 435/6.11; 435/306.1; 435/325

(57) ABSTRACT

The present invention relates to an integrated method for enriching and detecting rare cells in biological body fluid sample. The enriching method comprises: (a) removing plasma protein by centrifugation; (b) optionally adding a red cell lysis solution to carry out red cell lysis so as to remove the red blood cells; (c) adding immunomicrospheres or immunoadsorbent to incubate; and (d) carrying out density centrifugation based on a special cell separation medium for separating the circulating rare cells, residual red blood cells after removing red blood cells and the white blood cells combined on the immunomicrospheres. The method for detecting the enriched rare cells according to the present invention comprises combining immunohistochemistry based staining with immunofluorescence, or bicolor, tricolor or multicolor staining based on immunohistochemistry, and observing and identifying by fluorescence or ordinary optical microscope or a scanner based on microscope principle. The novel and unique method for enriching and staining has been proved to have low cost and can rapidly, effectively and high specifically enrich and quantitatively detect the rare cells in blood.

### FIELD OF THE INVENTION

The present invention generally relates to an integrated method for enriching and detecting rare cells from biological body fluid sample.

### BACKGROUND OF THE INVENTION

Since the technology of directly capturing the circulating tumor cells in the human peripheral blood of the U.S. Immunicon/Veridex (Philadelphia, USA) was examined and approved by U.S. Food and Drug Administration in 2004, important scientific researches and clinical significance on obtaining and detecting circulating tumor cells, circulating endothelial cells, tumor stem cells and some immune cells have been continuously and widely reported (Cristofanilli et al, 2004 New Eng J. Med. 351:781; Braun and Marth, 2004 New Eng. J. Med. 351:824).

However, such method for directly capturing the circulating tumor cells by using the antibody-coupled magnetic beads has some well-known disadvantages (Mocellin et al, 2006 Trends in Molecular Medicine 12:130): due to the heterogeneity of the expression of markers on tumor cells surface, a lot of tumor cells cannot be captured with this method, which has been proved by a lot of clinical cases; in addition, as the tumor cells are “contacted” and stimulated by the magnetic beads coupled to the antibody, these tumor cells that are captured and enveloped by a great deal of immune granules are caused to be no longer cells in a natural state, thus, it is hard to make subsequent analysis and research thereto. Consequently, people start to look for some other alternative means to obtain the circulating rare cells. Compared with the technology of directly capturing the cells, it has been well recognized that a method of enriching the circulating rare cells by removing red blood cells and white blood cells is the most efficient and most practicable alternative means. Although some single experimental methods for removing or separating specific cell population have been reported, such as density centrifugation method (U.S. Pat. No. 4,927,750), immune magnetic granule method (U.S. Pat. No. 4,177,145), red cell lysis method (hemolysis method), and a method of primary combining immune magnetic granule with density centrifugation which must utilize a special cell separation tube (U.S. Pat. No. 5,840,502), all these methods have been proved to be time-consuming, with a low removal rate of white blood cells and red blood cells and a low recovery rate of target cells, as well as inconvenience in operation brought by some special equipments required. Owing to the above reasons, several unrelated existing technologies are optimized and combined in the present invention so as to provide a set of novel and unique integrated method, comprising removing plasma protein, adding red cell lysis solution (hemolysis solution), adding antibody-coated immunomicrospheres, and a density centrifugation separation method based on a special cell separation medium. The unpredictable experimental result of this unique integrated experimental method has been proved to remove plasma protein, white blood cells and red blood cells quite rapidly, effectively and high specifically so that the object of effectively enriching the circulating rare cells is achieved and a high recovery rate of rare cells can be continuously maintained.

So far, methods of detecting the circulating rare cells, including the circulating tumor cells and residual white blood cells, in the enriched sample are all based on immunofluorescence staining. However, the inevitable high nonspecific staining signal, expensive fluorescence microscope and indispensable but inconvenient working environment (e.g. darkroom) greatly limit the development of detecting the circulating tumor cells and circulating endothelial cells based on immunofluorescence. The present invention provides a bicolor or multicolor staining method based on a specific combination of alkaline phosphatase and peroxidase labelled antibody, so as to achieve the object of detecting the enriched circulating rare cells. The circulating rare cells stained with this new method have good cell morphologies, and can be observed and analyzed by a ordinary optical microscope or a scanner; thereby, the enriched circulating rare cells can be rapidly and easily detected in the residual white blood cells.

### SUMMARY OF THE INVENTION

The present invention provides a set of novel and unique integrated methods, comprising: removing plasma protein, lysing red cells, adding immunomicrospheres or immune materials to remove white blood cells, adding density centrifugation based on a special cell separation medium to separate the circulating rare cells in the biological body fluid sample. The present method consisting of concentrating and enriching can rapidly enrich the circulating rare cells in the biological body fluid specimens, e.g. in peripheral blood, and also has a high recovery rate. The enriched cells have a good cell morphology that can be used for image analysis. Moreover, most of the white blood cells in the patient specimen also can be effectively recovered for application in other researches and analysis, for instance, research of gene profile. In the present invention, no special equipment, e.g. cell separation tube or magnet, is required.

In the present invention, the biological body fluid specimens collected from human or animal includes, but not limited to, the following sources: peripheral circulating blood, umbilical cord blood, urine, semen, bone marrow, amniotic fluids, spinal cord and pleural effusion, ascites, sputum, treated and/or homogenized human or animal tissues, cultured human or animal cells.

The immunomicrospheres are formed by covalently or noncovalently coupling the antibody which can specifically recognize the white blood cell marker to the microspheres surface which may be or may not be chemically treated to be suitable to couple to proteins. The microspheres, with a diameter in the range of 10 nanometers and 100 microns, i.e. 10 nm-100 μm, comprise or partially comprise any one of the following ingredients: silica, dextran, sepharose, agarose, or sephadex. The microspheres for preparing the immunomicrospheres are magnetic or nonmagnetic.

In the present invention, the antibody for preparing the immunomicrospheres specifically recognize, but not limited to, the following white blood cell surface markers: CD3, CD31, CD34, CD45, CD50, CD69, CD84, or CD102, etc. In the process of preparing the immunomicrospheres, either the antibody recognizing any one of these CD molecules or a combination of antibodies recognizing any two or more of these CD molecules is covalently or noncovalently coupled to any solid surface suitable for being coupled, for instance, magnetic or nonmagnetic microspheres with a diameter between 10 nanometers and 100 microns (10 nm-100 μm).

In the present invention, the immunoadsorbent is prepared by covalently or noncovalently coupling any solid surface which is suitable for binding proteins and has been chemically treated or not, such as silicon glass slide, to a ligand or a specific monoclonal or polyclonal antibody including antibody against the white blood cell surface marker, such as CD45.

In the present invention, the specific gravity range of the special cell separation medium is 1.07256-1.07638 gramme/millilitre (gr/ml or gr/cm3) at 20° C. The density in this specific range is suited to separate almost all nucleated cells from red blood cells and immunomicrospheres. The cell separation medium includes any one or any two or more of the following reagent ingredients: polyvinylpyrrolidine coated colloidal silica; polysucrose plus sodium diatrizoate or derivatives thereof; nonionic polymer consisting of sucrose and epichlorohydrin; or any one sugar-containing solution, such as dextran or sucrose; iodinated small molecular compounds (such as metrizamide); or any protein solution. The specific gravity of the cell separation medium can be adjusted to be within the range of 1.07256-1.07638 gramme/millilitre (gr/ml or gr/cm³) at 20° C. by a buffer that has an osmotic pressure of 280-320 mOsm/kg H₂O and pH 6.8-7.8. The specific gravity of the immunomicrospheres is higher than that of the cell separation medium. The centrifugation based on the cell separation medium is carried out in the common commercialized centrifuge tube.

The method for enriching the rare cells in the biological body fluid in the present invention further comprises: collecting all supernatants above the deposited cells obtained from centrifugation based on the cell separation medium.

In the present method for enriching the rare cells in the biological body fluid, the step of lysing the red blood cells to remove the red blood cells is carried out prior to, after or while adding the immunomicrospheres or immunoadsorbent to incubate. If the present method does not comprise the step of adding a red cell lysis solution to carry out red cell lysis, the red blood cells can be separated and removed via a long-time centrifugation.

The circulating rare cells enriched with the present method can be applied to the following aspects: counting of the enriched circulating rare cells by immunofluorescence or immunohistochemistry plus a ordinary optical microscope or a visible light scanner; PCR; flow cytometer detection; gene expression profile analysis; protein expression profile analysis; enzymology assay; in vitro screening chemotherapeutic medicament for a tumor patient; establishing a chemotherapeutic scheme for a tumor patient and guiding prosecution of chemotherapy; evaluation of effects of using chemotherapeutic medicament to the tumor cells in a tumor patient and/or one or more antibodies used to treat tumor; in vivo or in vitro culturing the enriched rare cells; identifying and acknowledging markers on the existing or newly found tumor cell surface or in the cells on the enriched rare cells; application of the enriched rare cells to clinical treatment; monitoring tumor recurrence of the tumor patient; developing new medicaments for treating tumor; acting as auxiliary means for tumor diagnosis; physical examination of healthy population; and diagnosis and treatment of heart disease based on the circulating endothelial cells.

Up to now, technical means for detecting the circulating rare cells are all based on immunofluorescence. However, the main drawback of nonspecific combination with the target cells resulted from the negative charge of the fluorescence dye itself is inevitable. It seriously troubles people when distinguishing true and false positive staining signals. The present invention provides a whole set of optimized and novel multicolor staining method based on immunohistochemistry so as to avoid nonspecific staining brought by the immunofluorescence and allow the stained circulating rare cells to be detected by a ordinary optical microscope or a scanner based on microscope principle. This method has been proved to be a high specific, rapid and simple technical means and have low cost, and no longer needs any fluorescence dye or expensive fluorescence microscope.

The method for detecting the enriched rare cells in the present invention may further comprise chromosomal fluorescence in situ hybridization.

The bicolor staining refers to respectively staining a marker of the rare cells, such as one or more keratins, and a marker of the white blood cells, such as CD45, thereby the rare cells and the white blood cells are stained into different colors; the tricolor staining refers to staining the nucleus of the rare cells with another color on the basis of the bicolor staining, or staining another marker of the rare cells with a third color, wherein the staining includes incubation of the primary monoclonal or polyclonal antibody specifically recognizing the rare cell markers and the primary monoclonal or polyclonal antibody specifically recognizing the white blood cells with the enriched rare cells.

The primary monoclonal or polyclonal antibody specifically recognizing the rare cell markers and the primary monoclonal or polyclonal antibody specifically recognizing the white blood cell markers are respectively covalently coupled to different small molecules selected from the group comprising but not limited to, rhodamine, biotin, digoxigenin, Alexa Fluor series molecules, FITC, and Texas Red.

In some specific embodiments of the present invention, the primary monoclonal or polyclonal antibody that can recognize any one or any two or more of keratins 8, 18, 19 or broad spectrum keratins is used to recognize the circulating tumor cells exfoliating into blood from any solid tumor of an epithelial source. The other monoclonal or polyclonal antibody that can recognize the white blood cell surface marker CD45 is used for white blood cell staining to distinguish false positive.

The staining comprises adding secondary monoclonal or polyclonal antibody that is coupled to different enzymes and can specifically recognize the small molecules. The coupled enzymes are peroxidase, or alkaline phosphatase, wherein the alkaline phosphatase is used to detect the enriched rare cells.

The rare cells enriched with the method in the present invention can be stained on a glass slide or in a solution.

In some specific embodiments of the present invention, the primary antibodies against the markers of the rare cells and the primary antibodies against the markers of the white blood cells are incubated in an arbitrary order together with the enriched rare cells, or both the antibodies are prepared into a mixture and incubated with the enriched rare cells.

In the present invention, the rare cells or other cells both can be directly captured by the antibody covalently or noncovalently coupled to any suitable solid surface, and can be enriched with the enriching method in the present invention.

The staining method in the present invention further comprises a combined use of immunofluorescence staining and immunohistochemistry based staining as well as observation in visible light, wherein the immunofluorescence is for detecting the enriched circulating rare cells, while the immunohistochemistry based staining is for staining the white blood cell.

In a certain specific embodiment of the present invention, the primary antibody recognizing keratins is marked with fluorescence molecules, while the primary antibody against CD45 is marked with small molecules to be used for the visible light color reaction based on immunohistochemistry and catalyzed by peroxidase.

The unique combination of the two methods for enriching and staining in the present invention can greatly improve popularization and application of detecting the rare cells such as circulating tumor cells in blood. The novel and unique methods for enriching and staining have been proved to have low cost and can rapidly, effectively and high specifically enrich and quantitatively detect the rare cells in blood.

The present invention further relates to a method for detecting enriched rare cells, comprising carrying out chromosomal fluorescence in situ hybridization, and observing and identifying by fluorescence or ordinary optical microscope or a scanner based on microscope principle.

The object of the present invention further lies in a kit for enriching rare cells in biological body fluid, comprising a red cell lysis solution, immunomicrospheres or immunoadsorbent, and a special cell separation medium. The kit further comprises an instruction on how to use the kit.

The present invention further relates to a kit for detecting enriched rare cells, comprising a primary monoclonal or polyclonal antibody specifically recognizing the rare cell markers and covalently coupled to small molecules, a secondary monoclonal or polyclonal antibody that can recognize the small molecules and is coupled to an enzyme, and a corresponding substrate of the enzyme. The kit optionally comprises an antibody marked with an immunofluorescence dye. It further comprises a probe and reagent for chromosomal fluorescence in situ hybridization. It further comprises an instruction on how to use the kit.

The present invention further relates to an automatic system for enriching circulating rare cells in biological body fluid sample, comprising a centrifuge for automatically removing plasma protein, a device for automatically adding a red cell lysis solution, a device for automatically adding immunomicrospheres or immunoadsorbent, a device for automatically adding a cell separation medium, a density centrifuge device and a device automatically collecting supernatants.

The present invention further relates to an automatic system for detecting enriched rare cells, comprising a bicolor or multicolor staining device based on immunohistochemistry, and an ordinary optical microscope or an automatic scanning device based on microscope principle. The staining device comprises an automatic sampling apparatus, an incubator and an automatic cleansing device.

Definitions

Rare cells: the proportion of the rare cells in all nucleated cells in the collected body fluid sample is less than 0.1%. They comprise circulating tumor cells, circulating endothelial cells, tumor stem cells, stem cells and some immune cells, etc.

Circulating rare cells: the circulating rare cells refer to the rare cells present in body fluid.

Biological body fluid specimens: they are fluids collected from human or animal body, including, but not limited to, the following sources: peripheral circulating blood, umbilical cord blood, urine, semen, bone marrow, amniotic fluid, spinal cord and pleural fluid, ascites, sputum, treated human or animal tissue, cultured human or animal cell.

Red cell lysis (hemolysis): lysing the red blood cells in a hypotonic condition.

Immunohistochemistry (IHC): showing the color observable under the optical microscope by reaction of substrate with enzyme coupled to the antibody.

Immunofluorescence: marking the antibody with fluorescence molecules.

### BRIEF DESCRIPTION OF THE DRAWINGS

FIG. 1 is an image obtained after tricolor staining of the circulating tumor cells with the present method based on immunohistochemistry in peripheral blood of a breast cancer patient after the circulating tumor cells are enriched with the method of the present invention.

FIG. 2 is an image obtained by detecting the circulating tumor cells by the method of chromosomal fluorescence in situ hybridization.

### DETAILED DESCRIPTION OF EMBODIMENTS

In the present invention, four unrelated single experimental means (i.e. removal of plasma protein, red blood cells lysis, antibody-coated immunomicrospheres and density centrifugation based on a special cell separation medium) are improved and optimally combined for the first time so as to provide a set of novel and unique methods that can rapidly and effectively enrich the circulating rare cells in peripheral blood or other body fluid samples. The enriched circulating rare cells are bicolor or multicolor stained by the technology derived from optimization of immunohistochemistry of the present invention, without immunofluorescence staining, and the observation, image acquisition, analysis and treatment of the stained rare cells can be completed by a ordinary optical microscope or a scanner. The rare cells include circulating tumor cells, circulating endothelial cells, tumor stem cells, stem cells and some immune cells, wherein the circulating tumor cells derives from any solid tumor of an epithelial source or not, e.g. melanoma.

1. The Method and Reagent for Enriching the Circulating Rare Cells Including Circulating Tumor Cells and Circulating Endothelial Cells in Blood

The technical means in the present invention can enrich or separate any desired rare cells from in vivo or in vitro body fluid specimens. The body fluid specimens include, but not limited to, the following sources: peripheral circulating blood, umbilical cord blood, urine, semen, bone marrow, amniotic fluid, spinal cord and pleural fluid, ascites, sputum, treated human or animal tissue, cultured human or animal cell.

In some specific embodiments of the present invention, the blood is collected in any one of commercialized blood collection tubes (e.g. BD, New Jersey, USA; Cyto-Chex, Iowa, USA). These blood collection tubes have any one of the following anticoagulants: citrate dextrose (ACD), ethylene diamine tetraacetic acid (EDTA), heparin, etc. The specimens should be treated within 72 hours.

In some other specific embodiments of the present invention, removing plasma protein, lysing red blood cells, adding antibody-coated immune magnetic beads and density centrifugation based on a special cell separation medium are combined and optimized in the present invention so as to effectively remove plasma protein, red blood cells and white blood cells. As an alternative option, the enriching step also can be simplified to consist of two steps, i.e. adding immunomicrospheres plus lysing red blood cells; or adding immunomicrospheres plus density centrifugation based on the special cell separation medium. All supernatants above the deposited cells are collected in the present invention, differently from other conventional practice of inaccurately collecting the boundary phase solutions of different specific gravities that is time consuming and needs efforts after separating the cells using the density centrifugation method.

In the embodiments of the present invention, the immunomicrospheres are prepared by covalently or noncovalently coupling the monoclonal or polyclonal antibody to any solid surface which is suitable for binding proteins and has been chemically treated or not (e.g. microspheres with a diameter of 10 nm-100 μm). These microspheres include or partially include any one of the following ingredients: silica, dextran, sepharose, agarose, or sephadex. These microspheres may be magnetic or nonmagnetic.

In some embodiments of the present invention, the immunomicrospheres can be replaced with immunoadsorbent that is prepared by covalently or noncovalently coupling the specific monoclonal or polyclonal antibody to any solid surface which is suitable for binding protein and has been chemically treated or not, such as silicon glass slide.

In some embodiments of the present invention, the special cell separation medium for density centrifugation has a specific gravity within a particular range, i.e. 1.07256-1.07638 gramme/millilitre (gr/ml or gr/cm³). The cell separation medium within this specific gravity range can be used to separate the desired cells. The cell separation medium in the present invention includes any one or any two or more of the following reagent ingredients: polyvinylpyrrolidine coated colloidal silica; polysucrose plus sodium diatrizoate or derivatives thereof; nonionic polymer consisting of sucrose and epichlorohydrin; or any one sugar-containing solution, such as dextran or sucrose; iodinated small molecular compounds (such as metrizamide); and/or any protein solution. The specific gravity of the cell separation medium can be adjusted by any buffer that has an osmotic pressure of 280-320 mOsm/kg H₂O and pH 6.8-7.8.

The specific gravity of the immunomicrospheres is higher than that of the cell separation medium.

In the implementation method of the present invention, the plasma protein can be removed by centrifugation.

In the present invention, the red cell lysis method and density centrifugation based on a special cell separation medium are combined for the first time so as to rapidly and effectively remove the red blood cells.

In the present invention, the immunomicrospheres and density centrifugation based on a special cell separation medium are combined for the first time so as to rapidly and effectively remove the white blood cells. As an alternative, removing the white blood cells in the present invention also can be simplified to only using immunomicrospheres or immunoadsorbent.

In the specific embodiments of the present invention, the antibody for preparing the immunomicrospheres or immunoadsorbent can be an antibody specifically recognizing any following white cell surface markers or an antibody recognizing any two or more of the following white cell surface markers: CD3, CD31, CD34, CD45, CD50, CD69, CD84, or CD102, etc.

In the specific embodiments of the present invention, removal of the red blood cells and white blood cells can be carried out in any suitable order. They can be removed simultaneously, or either the red blood cells or the white blood cells can be removed first.

The enriched circulating rare cells can be used for a series of subsequent analysis, including immunofluorescence analysis, staining analysis based on immunohistochemistry, PCR, in vivo or in vitro culturing the enriched circulating rare cells, etc.

2. Detecting the Enriched Circulating Rare Cells Including Circulating Tumor Cells

Up to now, all the published methods relating to detecting the circulating tumor cells are based on immunofluorescence staining. However, the inevitable main drawback of immunofluorescence staining, i.e. nonspecific staining known as “ghost”, seriously troubles people in judging true and false positive cells. The present invention provides a whole set of optimized multicolor staining methods based on immunohistochemistry. Nonspecific staining can be greatly eliminated after the circulating rare cells enriched with the experimental means in the present invention are stained with this method. Combination of this staining method with the ordinary optical microscope makes it quite convenient for people in different fields to develop detections of circulating rare cells.

In a certain specific embodiment of the present invention, the circulating rare cells enriched with the present method are fixed by 2% of paraformaldehyde.

In other specific embodiments of the present invention, the primary monoclonal or polyclonal antibody that can recognize any one or any two or more of keratins 8, 18, 19 or broad spectrum keratins is used to recognize the circulating tumor cells, which, in blood, exfoliate from any solid tumor of epithelial source. The other monoclonal or polyclonal antibody that can recognize the white blood cell surface marker CD45 is used to distinguish false positive.

In some other specific embodiments of the present invention, the primary monoclonal or polyclonal antibody against keratins or CD45 is respectively covalently coupled to any one of the following small molecules including, but not limited to, rhodamine, biotin, digoxigenin, Alexa Fluor series molecules, FITC, and Texas Red, etc.

In the other specific embodiments of the present invention, the secondary monoclonal or polyclonal antibody that can specifically recognize the small molecules marked on the primary antibody is covalently coupled to alkaline phosphatase, peroxidase or other enzymes, respectively.

In a certain specific embodiment of the present invention, as another alternative, the immunofluorescence can be combined with immunohistochemistry identified by visible light. In this method, the primary monoclonal or polyclonal antibody, which can recognize keratin, is marked with fluorescence molecules of any color, such as Alexa Fluor series, Quantum dot, FITC, etc., while the primary antibody against CD45 is marked with the above small molecules to be used in the immunohistochemistry visible light color reaction catalyzed by peroxidase. This combination can greatly reduce the nonspecific staining of the white cell surface marker CD45 caused by immunofluorescence.

The automatic staining device comprises an automatic sampling apparatuse, an incubator and an automatic cleansing device.

### EXAMPLES

### Example 1

### Enriching the Circulating Tumor Cells in Peripheral Blood of a Breast Cancer Patient

5 ml of human peripheral blood is collected in a blood collection tube (BD, New Jersey, USA) containing ethylene diamine tetraacetic acid (EDTA) anticoagulant. The supernatant can be absorbed out with a pipette or automatic liquid-absorption device so as to remove plasma proteins after the blood samples are centrifuged (700×g, 10 minutes). The deposit obtained after centrifugation is resuspended in 30 ml of a red cell lysis solution (BD Pharmingen, California, USA) and incubated for 20 minutes. The specimen centrifugation is carried out (700×g, 10 minutes) so as to separate the lysed red blood cell chips in the supernatant. The deposit (i.e. deposited cells) is resuspended in 5 millilitre of phosphate buffer (pH 7.4) after the supernatant is removed. 0.5 millilitre of magnetic beads coated with a monoclonal antibody against white blood cell surface antigen such as CD45 (Invitrogen, California, USA) is added thereto to incubate for 30 minutes at a room temperature. All the reaction solutions are added to the top layer of 5 millilitre of the cell separation medium in a common 50 millilitre centrifuge tube for centrifugation 10 minutes, 400×g. All supernatants are collected. The supernatants are centrifuged 900×g for 10 minutes. The deposited cells obtained after centrifugation can be used for further analysis after resuspended in phosphate buffer.

The cell separation medium in this example is prepared by adjusting the density of a mixture of 5.7% of polysucrose and 9% of sodium diatrizoate (Sigma, Missouri, UDA) by PBS to 1.07256-1.07638 gramme/millilitre (gr/ml or gr/cm³) under monitoring of a high precision digital density meter (model: DMA 4500, Anton-Paar, Virginia, USA) at 20° C.

### Example 2

### Tricolor Staining the Circulating Tumor Cells Enriched in Peripheral Blood of a Breast Cancer Patient

The enriched circulating tumor cells are put on the glass slide and fixed by 2% of paraformaldehyde prepared from phosphate buffer for 2 hours at room temperature, followed by washing thrice with phosphate buffer. The cells and a mixture (diluted by phosphate buffer) containing biotin (Pierce, Ill., USA) labelled monoclonal antibody (Abcam, UK, 1 μg/ml) against keratins 8+18+19 and rhodamine (Pierce, Ill., USA) labelled monoclonal antibody (Abcam, UK, 1 μg/ml) against CD45 are incubated for 30 minutes at a room temperature. After the glass slide is washed thrice with the phosphate buffer, it is incubated for 30 minutes at the room temperature with a mixture (diluted by phosphate buffer) containing alkaline phosphatase labelled monoclonal antibody (Sigma, Missouri, USA, 1 μg/ml) against biotin and peroxidase (Pierce, Ill., USA) labelled monoclonal antibody (Abcam, UK, 1 μg/ml) against rhodamine After the glass slide is washed thrice with the phosphate buffer, the color reaction is carried out using Nuclear Fast Red kit produced by Vector Laboratories (California, USA), alkaline phosphatase and peroxidase substrate kit. See the accompanying figure for the staining result.

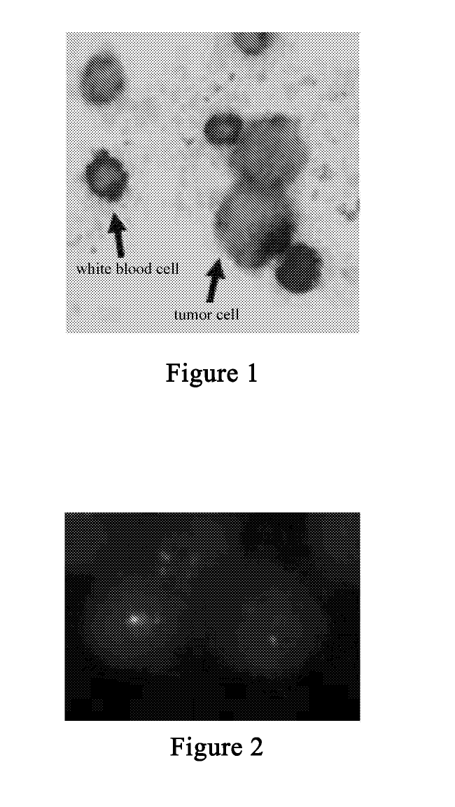

With reference to FIG. 1, the circulating tumor cells in peripheral blood of the breast cancer patient are stained with the tricolor staining method based on immunohistochemistry after they are enriched with the experimental method in the present invention. The figure shows the circulating tumor cells observed under a ordinary optical microscope. Big cells: breast cancer cells (tumor cell), wherein keratins being stained into blue and nucleus into pink; and small cells: white blood cells (WBC), in which the surface CD45 is stained into brown.

### Example 3

### Detecting the Circulating Tumor Cells by a Chromosomal Fluorescence in situ Hybridization

The enriched tumor cells are put on the glass slide as specimens. The glass is rinsed with SSC buffer after the stained specimens are treated with 20 milligramme/millilitre of RNA enzyme for 1 hour. The specimens are dehydrated with absolute ethyl alcohol for 10 minutes and then heated to 70° C., holding for 5 minutes for denaturation. The specimens are dehydrated with absolute ethyl alcohol for 10 minutes again, and hybridized and incubated with a probe at 45° C. overnight. The specimens are observed by a fluorescence microscope after being washed with the SSC buffer. The specimens can be the enriched tumor cells stained with the method in Example 2. The object of carrying out chromosomal fluorescence in situ hybridization is further confirming authenticity of detecting the tumor cells by tricolor staining based on immunohistochemistry. For the sake of rapid diagnosis, the specimens may not be stained by the antibody but chromosomal fluorescence in situ hybridization is directly carried out. See FIG. 2 for the result of chromosomal fluorescence in situ hybridization. The chromosomes of the cells are shown to be red and green. Based on the number of the red or green, a judgment can be made whether the chromosomes vary and whether they are tumor cells.

### Example 4

### Applying the Circulating Tumor Cell Detection to Rapidly and Clinically Evaluate the Curative Effect of Anti-Tumor Chemotherapeutical Medicament and Monitor Tumor Recurrence

At present, the conventional method for clinically evaluating the curative effect of chemotherapeutical medicament is to make CT examination for the patient every three months. Such a long time interval fatally harms those patients whose chemotherapeutical effects are unfavorable. But the detection of the circulating tumor cells every 1-2 weeks can provide doctor accurate evaluation data 2-4 weeks just after chemotherapy starts. The decreased circulating tumor cell number indicates validity of the chemotherapeutical medicament. Contrarily, if the circulating tumor cell number does not obviously change or even increases, it means the patient need to accept different chemotherapeutical medicament treatments.

Tumor recurrence means the primary or metastasis tumor comes into an active stage again. At this moment, the circulating tumor cell number in blood of the patient will rise prominently. Quite convincing evidences can be provided for judging whether tumor recurs at an early stage by making a long-term trail observation (usually one examination every three months) of the circulating tumor cells for the tumor patient leaving hospital after treatment.

It would be understood by the person skilled in the art that the above preferred examples are only to illustrate the present invention but not to limit the present invention. Various improvements, combinations, sub-combinations and alterations thereto can be made as needed. All improvements, combinations, sub-combinations, alterations and equivalent substitutions fall into the scope of the appended claims.

1. An integrated method for enriching rare cells in biological body fluid sample, comprising:
(a) removing plasma proteins by centrifugation,
(b) optionally adding a red cell lysis solution to carry out red cell lysis so as to remove red blood cells,
(c) adding immunomicro spheres or immunoadsorbent to incubate,
(d) carrying out density centrifugation based on a special cell separation medium to separate the rare cells, residual red blood cells after removing the red blood cells and white blood cells combined on the immunomicrospheres.

2. The method according to claim 1, wherein the biological body fluid specimen collected from human or animal comprises, but not limited to, the following sources: peripheral circulating blood, umbilical cord blood, urine, semen, bone marrow, amniotic fluids, spinal cord and pleural fluid, ascites, sputum, treated and/or homogenized human or animal tissue, cultured human or animal cell.

3. The method according to claim 1, wherein the immunomicrospheres are formed by covalently or noncovalently coupling an antibody specifically recognizing a white blood cell marker to the microspheres surfaces, which are or are not chemically treated so as to be suitable for coupling with proteins; wherein the microspheres, with a diameter between 10 nanometers and 100 microns, comprise or partially comprise any one of the following ingredients: silica, dextran, sepharose, agarose, or sephadex.

4. The method according to claim 3, wherein the microspheres for preparing the immunomicrospheres are magnetic or nonmagnetic.

5. The method according to claim 1, wherein the immunoadsorbent is prepared by covalently or noncovalently coupling any solid surface which is suitable for binding the proteins and has been chemically treated or not, such as silicon glass slide, to a ligand or a specific monoclonal or polyclonal antibody including antibody against a white blood cell surface marker, such as CD45.

6. The method according to claim 1, wherein a specific gravity range of the special cell separation medium is 1.07256-1.07638 gramme/milliliter at 20° C., the cell separation medium includes any one or any two or more of following reagent ingredients: polyvinylpyrrolidine coated colloidal silica; polysucrose plus sodium diatrizoate or derivatives thereof; nonionic polymer consisting of sucrose and epichlorohydrin; or any one sugar-containing solution, such as dextran or sucrose; iodinated small molecular compounds (such as metrizamide); or any protein solution, the specific gravity of the cell separation medium can be adjusted to be within the range of 1.07256-1.07638 gramme/millilitre at 20° C. by a buffer that has an osmotic pressure of 280-320 mOsm/kg H₂O and pH 6.8-7.8.

7. The method according to claim 1, wherein the specific gravity of the immunomicrospheres is higher than the specific gravity of the cell separation medium.

8. The method according to claim 1, wherein the centrifugation based on the cell separation medium is carried out in a common commercialized centrifuge tube.

9. The method according to claim 1, further comprising collecting all supernatants above deposited cells obtained from the centrifugation based on the cell separation medium.

10. The method according to claim 1, wherein the step of lysing red blood cells to remove the red blood cells is carried out prior to, after or while adding the immunomicrospheres or immunoadsorbent to incubate.

11. The method according to claim 1, wherein the rare cells refer to those occupying a proportion in all nucleated cells from collected biological body fluid sample of less than 0.1%, and the rare cells comprise circulating tumor cells, circulating endothelial cells, tumor stem cells, stem cells and some immune cells; wherein the circulating tumor cells come from any solid tumor of an epithelial source or not, e.g. melanoma, etc.

12. A use of the rare cells enriched with a method, which comprises (a) removing plasma proteins by centrifugation, (b) optionally adding a red cell lysis solution to carry out red cell lysis so as to remove red blood cells, (c) adding immunomicrospheres or immunoadsorbent to incubate, (d) carrying out density centrifugation based on a special cell separation medium to separate the rare cells, residual red blood cells after removing the red blood cells and white blood cells combined on the immunomicrospheres, in following aspects: counting of the enriched rare cells by immunofluorescence or immunohistochemistry plus a fluorescence or ordinary optical microscope or a visible light scanner; PCR; detecting of a flow cytometer; analysis of gene expression profile; analysis of protein expression profile; enzymology assay; in vitro screening chemotherapeutic medicament for a tumor patient; establishing a chemotherapeutic scheme for a tumor patient and guiding prosecution of chemotherapy; evaluation of the effects of using chemotherapeutic medicament to the tumor cells in the tumor patient and/or one or more antibodies used to treat tumor; in vivo or in vitro culturing the enriched rare cells; identifying and acknowledging markers on existing or newly found tumor cell surface or in the cells on the enriched rare cells; application of the enriched rare cells to clinical treatment; monitoring tumor recurrence of a tumor patient; developing new medicaments for treating tumor; acting as auxiliary means for tumor diagnosis; physical examination of healthy population; and diagnosis and treatment of heart disease based on circulating endothelial cells.

13-23. (canceled)

24. A kit for enriching rare cells in biological body fluid, comprising a red cell lysis solution, immunomicrosphere or immunoadsorbent, and a cell separation medium.

25. The kit according to claim 24, further comprising an instruction on how to use the kit.

26-29. (canceled)

30. An automatic system for enriching circulating rare cells in biological body fluid sample, comprising a centrifuge for automatically removing plasma protein, a device for automatically adding a red cell lysis solution, a device for automatically adding immunomicrospheres or an immunoadsorbent, a device for automatically adding a cell separation medium, a density centrifuge device and a device for automatically collecting supernatants.

31-32. (canceled)
